(19) United States
(12) Patent Application Publication
(10) Pub. No.: US 2011/0144213 A1
(43) Pub. Date: Jun. 16, 2011

(54) ANHYDROUS DEPIGMENTING COMPOSITIONS COMPRISING A SOLUBILIZED PHENOLIC COMPOUND

(76) Inventors: Claire Mallard, Mougins (FR); Karine Nadau-Fourcade, Villeneuve-Loubet (FR); Fabienne Louis, Villeneuve-Loubet (FR)

(21) Appl. No.: 12/994,908

(22) PCT Filed: Jun. 2, 2009
(86) PCT No.: PCT/FR2009/051038

§ 371 Date: Feb. 28, 2011

(30) Foreign Application Priority Data
May 30, 2008 (FR) 0853578

(51) Int. Cl. A61K 31/05; A61K 31/085; A61P 17/00; A61Q 19/00; A61K 8/18; A61K 8/34

(52) U.S. Cl. 514/721; 514/731; 514/718

(57) ABSTRACT

Novel anhydrous dermatological depigmenting compositions, especially for topical application, contain, as pharmaceutical active agent, a phenolic compound dissolved in a fatty phase therefor.

The present invention relates to a novel cosmetic or pharmaceutical depigmenting composition, characterized in that it comprises, as pharmaceutical active agent, a phenolic derivative dissolved in the fatty phase, for topical application, and to the process for preparing it and to its use in dermatology.

Among the therapeutic agents recommended in the treatment of cutaneous hyperpigmentation, phenolic derivatives and more particularly polyphenols have for decades been among the active agents that are the most effective. The therapeutic use of these agents results from the observation of cutaneous depigmentation in the case of operatives in the rubber industry, in which some of these products are used as antioxidants. Subsequently, numerous studies have confirmed their efficacy, alone or combined with other depigmenting agents [Jorge L. Sanchez, M.D. and Miguel Vazquez, M.D. International Journal of Dermatology January-February 1982 Vol. 21, pp. 55-58]. They are thus found to be active agents that are virtually indispensable in the treatment of hyperpigmentation and are consequently present in many commercial products.

Among the phenolic derivatives, polyphenols such as, especially, hydroquinone are the pharmaceutical active agents most commonly used. Hydroquinone has been the subject of filing of various patent applications, and in particular U.S. Pat. No. 3,856,934 in which hydroquinone is in combination with retinoic acid and a corticoid as a depigmenting composition.

Rucinol or lucinol, or 4-butylresorcinol, is also a phenolic-based pharmaceutical active agent, of polyphenol type, sold as an agent for lightening brown marks associated with pigmentation disorders (the product Iklen®).

However, in the majority of cases, hydroquinone, rucinol or salts or derivatives thereof are dissolved in the aqueous phase of the preparation.

It is known that a certain number of active principles with advantageous therapeutic activity are sensitive to oxidation and especially undergo chemical degradation leading to a substantial loss of their activity in the presence of water. The incorporation of a phenolic derivative such as hydroquinone or rucinol is thus a major drawback in this type of aqueous preparation.

Specifically, degradation of formulations containing phenolic derivatives such as hydroquinone or rucinol, alone or in combination with other active principles, is often observed. These active agents are effectively known for their great sensitivity to oxidation and to heat, leading to a reduction in efficacy, rapid browning of the formulations and occasionally even demixing of the formulation.

Furthermore, to accelerate their solubilization, phenolic derivatives such as hydroquinone or rucinol are often exposed to heat during the phase of preparing the composition, especially in standard emulsions, and this phenomenon initiates and accelerates the browning.

In the prior art, reducing agents are used to combat this degradation, in particular sulfites, which are virtually indispensable. However, these antioxidants have a certain number of drawbacks, such as skin irritation problems, odour of the formulations or destabilization of the formula associated with a loss of viscosity.

Another drawback due to the presence of phenolic derivatives such as hydroquinone or rucinol, alone or in combination with other active agents in the composition, is their strong irritant power.

As a result of its irritant power, hydroquinone at high concentration can give rise to post-inflammatory hypermelanosis and ochronosis phenomena.

Local irritation and dermatitis may develop after a prolonged use of hydroquinone at high concentration [“N-acetyl4S cysteaminylphenol as a new type of depigmenting agent” Jimbow K. Arch. Dermatol. 1991 October; 127 (10): 1528-1534].

Treatment with hydroquinone may be accompanied by irritation that may lead to a post-inflammatory hyperpigmentation. The incidence of the irritation depends on the hydroquinone concentration. This irritation is relatively high for 10% concentrations and reduces greatly for preparations with a 5% dose, and is considered to be virtually nonexistent at a concentration of 2% [“Les agents chimiques dépigmentants (Depigmenting chemical agents)” JP. Ortonne Ann. Dermatol. Venerol. 1986, 113: 733-736].

The chosen galenical form may thus play a predominant role in minimizing these effects.

Consequently, phenolic derivatives and in particular hydroquinone or rucinol should be formulated in solubilized form in a formulation, which allows the presence of sulfites to be avoided and which allows the use of antioxidants to be limited to a minimum.

In the anhydrous compositions described in the prior art, hydroquinone is generally dissolved in alcoholic or glycolic solvents before being incorporated into the rest of the anhydrous preparation.

This is the case especially in patent application US 2006/0 120 979, which describes a composition comprising hydroquinone and an anhydrous base formed from an anhydrous solvent and a high molecular weight silicone vehicle. In this case, the hydroquinone is dissolved in a solvent preferably selected from the group of monohydric alcohols (such as isopropanol), dihydric alcohols (such as glycols) and trihydric alcohols (such as glycerol). These compositions do not contain any sulfites, but require lipophilic anti-oxidants in relatively large amount. The reason for this is that, in such a medium, hydroquinone nevertheless undergoes degradation, which is less pronounced than in water but substantial enough to require the presence of lipophilic antioxidants in proportions ranging up to 0.75% by weight relative to the weight of the composition.

U.S. Pat. No. 4,466,955 also discloses compositions of anhydrous type containing hydroquinone. The solvents used are only solvents of polyalkoxylated fatty acid ether type (PPO or PEO derivatives). Moreover, these solvents must be used at a large concentration of between 30-60% (preferably 40-45%) and not lower under any circumstances, in order to manage to dissolve between 2-10% of hydroquinone. Moreover, despite the choice of these solvents, degradation of the hydro-quinone is observed if rapid cooling is not performed. Moreover, it is pointed out that the heating temperature of the phase containing the hydroquinone should not be greater than 45° C. This thus places considerable constraints on the manufacturing processes.

One of the aims of the present invention here is to dissolve the phenolic derivative in an oily solvent in which the active agent is both soluble and stable and in which it is then possible to envisage the incorporation of the active agent into manufacturing processes that require heating steps without having any impact on the stability of the active agent.

Another aim of the present invention is to propose an anhydrous pharmaceutical composition for topical application that has prolonged stability, allowing optimized release of the active agent while at the same time being very well tolerated.

The present invention thus relates to a novel anhydrous stable composition, especially for topical application, comprising a phenolic derivative of polyphenol type dissolved in the fatty phase.

By virtue of its anhydrous composition, the composition according to the invention ensures both excellent stability and harmlessness of the composition.

One subject of the present invention is an anhydrous pharmaceutical composition comprising a pharmaceutical active agent of phenolic derivative type, and especially of polyphenol type, and characterized in that the said phenolic derivative is dissolved in the fatty phase.

As pharmaceutical active agents of phenolic derivative type according to the invention, mention may be made in a non-limiting manner of polyphenols and more particularly hydroquinone, rucinol or lucinol and salts thereof, 4-hydroxyanisole, hydroquinone monoethyl ether and hydroquinone monobenzyl ether. Hydroquinone, or rucinol and salts thereof, is preferably used. The term “rucinol salts” especially means salts formed with a pharmaceutically acceptable base, especially a mineral base such as sodium hydroxide, potassium hydroxide and aqueous ammonia, or an organic base such as lysine, arginine or N-methylglucamine, but also the salts formed with fatty amines such as dioctylamine, aminomethylpropanol and stearylamine.

Advantageously, the amount of phenolic derivative is from 0.01% to 10% by weight, preferably from 0.05% to 6% by weight and more particularly from 0.1% to 5% by weight relative to the total weight of the composition.

The term “anhydrous composition” means a composition comprising an amount of water of less than or equal to 5% by weight relative to the total weight of the composition. In one preferred mode according to the invention, the composition does not contain any water.

The term “stable composition” means a chemically and physically stable composition.

The term “chemical stability” especially means that no degradation of the active agent is observed over time and at temperatures between 4 and 40° C. The term “physical stability” especially means that the compositions do not show any changes in macroscopic appearance, in particular of colour, or in microscopic appearance, and no change in viscosity over time and at temperatures of between 4 and 40° C.

Throughout the present patent application, the term “room temperature” means a temperature between 20 and 30° C.

The anhydrous nature of the composition according to the invention makes it possible to avoid the instability of the phenolic derivative, in particular its oxidation in aqueous medium. In such a formulation, the use of sulfites, which are indispensable for stabilizing hydroquinone or rucinol in aqueous medium, is thus no longer necessary. The compositions according to the invention make it possible to avoid the use of sulfites and to reduce the amount of antioxidants conventionally used in compositions containing water.

In one preferred mode according to the invention, the composition does not contain any sulfites and contains an amount of antioxidants strictly less than 0.3% by weight relative to the total weight of the composition. The antioxidants that may be used according to the invention are preferably antioxidants such as vitamin E and derivatives thereof, for instance DL-α-tocopherol or tocopheryl acetate from Roche; vitamin C and derivatives thereof, for instance ascorbyl palmitate from Roche, and the butylhydroxy-toluene sold under the name Nipanox BHT by Clariant.

In one particularly preferred mode, the composition according to the invention does not contain any antioxidant.

In one particularly preferred mode, the composition according to the invention does not comprise any preserving agent. Specifically, by virtue of its anhydrous nature and given the choice of ingredients, the composition according to the invention is a self-protected formulation. According to the invention, the term “self-protected” refers to a formulation that does not require the presence of a preserving agent in order to ensure its bacteriological cleanliness. The absence of preserving agent ensures the absence of known intolerance or sensitization that might be induced by the preserving agents.

The composition according to the invention comprises at least one fatty phase that is a solvent for the active agent, or an oily phase that is a solvent for the active agent, allowing the desired quality of solubilities and stabilities of the active agent to be obtained.

The term “oily solvents for the active agent” especially refers to:
plant oils, such as castor oil, the sweet almond oil sold by Sictia or the sesame oil sold by CPF;
silicone oils such as the cyclomethicone sold under the name ST-Cyclomethicone 5NF by Dow Corning or the dimethicone sold under the name Q7 9120 Silicone Fluid by Dow Corning;
mineral oils, such as Marcol 152 or Primol 352 sold by Esso;
perhydrosqualene;
triglycerides, such as the caprylic/capric triglycerides sold under the name Miglyol 812 N by IMCD, or derivatives such as PEG-8 caprylic/capric triglycerides sold under the name Labrosol by the company Gattefossé;
esters, such as the octyldodecyl myristate sold under the name MOD by Gattefossé, the C₁₂-C₁₅ alkyl benzoate sold under the name Tegosoft TN by Goldschmidt or the cetearyl isononanoate sold under the name Cetiol SN PH by Cognis, or the diisopropyl adipate sold under the name Crodamol DA by Croda;
Guerbet alcohols, such as the octyldodecanol sold under the name Eutanol G by Cognis;
ethers and derivatives, such as the PPG-15 stearyl ether sold under the name Arlamol E by Croda;
and mixtures thereof.

PPG-15 stearyl ether or any other ether or derivatives, diisopropyl adipate or any other ester of derivatives, or alternatively triglycerides such as caprylic/capric triglycerides or derivatives thereof, or a mixture of these compounds, will preferably be chosen as oily solvents for the active agent. The composition according to the invention more particularly comprises a mixture of solvents. Preferentially, the mixture of solvents will be formed from a maximum of 15% (by weight relative to the total weight of the composition) of solvent of ether derivative type. In the composition according to the invention, this amount of solvent, combined with the other novel solvents present, is sufficient to dissolve the desired concentrations of active agent and to obtain stable preparations.

In one mode according to the invention, the oily phase that is a solvent for the active agent comprises at least one oily solvent for the active agent and/or a lipophilic surfactant.

The term “lipophilic surfactant” more particularly means:
polyoxyethylenated castor oil derivatives, for instance PEG-35 castor oil sold especially under the name Cremophor EL by BASF;
polyoxyethylenated derivatives of fatty acid esters, for instance PEG-8 caprylic capric triglycerides sold under the name Labrasol by Gattefossé.

Preferably, the composition comprises at least one oily phase that is a solvent for the active agent. It may also comprise at least one fatty phase that is a non-solvent for the active agent. Preferably, the composition comprises an oily phase that is a solvent for the active agent and a fatty phase that is a non-solvent for the active agent; alternatively, preferably, the composition comprises only an oily phase that is a solvent for the active agent.

The amount of solvent fatty phase in the composition according to the invention is generally between 5% and 99% and preferentially from 10% to 98% by weight relative to the total weight of the composition.

According to one particular embodiment, the compositions according to the invention do not contain any alcoholic or glycolic solvents.

The composition according to the invention may also comprise at least one lipophilic gelling agent or thickener depending on the desired viscosity. Specifically, these compounds are used in the present invention as “viscosity regulators”.

According to the invention, the term “lipophilic thickeners or gelling agents” means compounds chosen especially from waxes, hydrogenated oils and fatty acid esters.

The term “wax” generally means a lipophilic compound, which is solid at room temperature (25° C.), with a reversible solid/liquid change of state, which has a melting point of greater than or equal to 30° C., which may be up to 200° C. and especially up to 120° C. As waxes that may be used, mention may be made of carnauba wax, microcrystalline waxes, beeswax, sold under the name Cerabeil blanche by Barlocher, glyceryl behenate, derivatives thereof such as glyceryl monobehenate, glyceryl dibehenate, tribehenine or a mixture thereof, such as that sold under the name Compritol 888 by Gattefossé, or candelilla wax.

The term “hydrogenated oil” means oils obtained by catalytic hydrogenation of animal or plant oils containing linear or branched C₈-C₃₂ fatty chains. Among these oils, mention may be made especially of hydrogenated jojoba oil, isomerized jojoba oil such as partially hydrogenated trans-isomerized jojoba oil manufactured or sold by the company Desert Whale under the commercial reference Iso-Jojoba-50®, hydrogenated sunflower oil, the hydrogenated castor oil sold especially under the name Cutina HR by Cognis, the polyoxyethylenated castor oil sold especially under the name Cremophor EL by BASF, hydrogenated coconut oil and hydrogenated lanolin oil; hydrogenated castor oil will preferably be used.

As fatty acid esters that may be used, mention may be made of lanolin sold especially under the name Medilan by Croda, the fatty acid esters of glycerol sold under the name Gelucire by Gattefossé, the hydrogenated coconut glycerides sold under the name Akosoft 36 by Karlshamns, or the diethylene glycol or propylene glycol monostearate sold, respectively, under the names Hydrine or Monostéol by Gattefossé.

The amount of lipophilic thickeners or gelling agents in the composition according to the invention is generally between 1% and 40% and preferably between 5% and 30% by weight relative to the total weight of the composition.

The composition according to the invention may contain an elastomer. The term “elastomer” means any polyorganosiloxane elastomer, i.e. any chemically crosslinked siloxane polymer that has viscoelastic properties especially such as, preferably, the Elastomer 10 sold by Dow Corning. The amount of high molecular weight elastomer in the composition according to the invention is generally between 0% and 40% and preferentially from 0 to 20% by weight relative to the total weight of the composition.

Optionally, the composition according to the invention may also comprise another surfactant and/or at least one binder.

The surfactants used are preferably nonionic surfactants, which are used for example, but not exclusively, to facilitate the incorporation of certain constituents such as glycols into the oily phase of the composition.

Among the surfactants that may be used according to the invention, mention may be made of esters of glycerol and optionally of polyethylene glycol, such as the mixture of glyceryl stearate and of PEG-100 stearate, sold under the name Arlacel 165 by Uniqema, the mixture of glyceryl stearate and of PEG-75 stearate sold under the name Gelot 64 by Gattefossé, the glyceryl stearate sold under the name Cutina GMSV by Cognis; emulsifying waxes, such as the self-emulsifying wax sold under the name Polawax NF by Croda or the PEG-8 beeswax sold under the name Apifil by Gattefossé; the polysorbate 80 sold under the name Tween 80 by Uniqema; the polyoxyethylenated castor oil from BASF sold especially under the trade name Cremophor EL or the mixture of glyceryl stearate and PEG-2 stearate, sold under the name Sedefos 75 by Gattefossé. The amount of surfactants in the composition according to the invention is between 0.1% and 10% by weight and preferably between 1% and 10% by weight.

The composition may also comprise at least one binder. Among the binders that may be used, mention may be made of the magnesium stearate sold by Brenntag, the corn starch sold by Roquette, the talc sold by WCD, the cholesterol sold by Croda or the silica sold by Degussa.

The binders may be used in an amount of between 0.1% and 30% by weight and preferably between 1% and 20% by weight.

The composition according to the invention may also contain additives, which a person skilled in the art will select as a function of the desired effect.

Among the additives, examples that may be mentioned include, taken alone or in combination:
vitamins such as vitamin PP or niacinamide;
calmatives or anti-irritant agents such as PPG-12/SMDI copolymer sold by the company Bertek Pharmaceuticals under the trade name Polyolprepolymer-2 or glycyrrhetinic acid or derivatives thereof, for instance Enoxolone sold by Cognis;
moisturizers or humectants: examples that may be mentioned include sugars and derivatives, glycols, glycerol and sorbitol;
lecithins and cholesterol;
preserving agents, such as the methyl paraben sold under the name Nipagin M by Clariant, the propyl paraben sold under the name Nipasol by Clariant, or the phenoxyethanol sold under the name Phenoxetol by Clariant;
acids or bases such as citric acid, sodium citrate, triethanolamine, aminomethylpropanol, sodium hydroxide and diisopropanolamine;
other additives for giving the said preparation specific properties.

Preferentially, the composition according to the invention comprises, on a weight basis relative to the total weight:
0.01% to 10% of at least one pharmaceutical active agent of phenolic derivative type,
0.05% to 99% of solvent oily phase and/or lipophilic surfactants,
0 to 50% of additional lipophilic gelling agents or thickeners,
0 to 20% of additives.

More preferentially, the composition according to the invention comprises, on a weight basis relative to the total weight:
0.05% to 6% of at least one phenolic derivative, of polyphenol type
1% to 99% of solvent oily phase and/or lipophilic surfactants,
1% to 40% of additional lipophilic gelling agents or thickeners,
0 to 20% of surfactants,
0 to 30% of binder(s),
0 to 10% of additives.

Even more preferentially, the composition according to the invention comprises, on a weight basis relative to the total weight:
0.01% to 5% hydroquinone or rucinol,
1% to 98% of solvent oily phase and/or lipophilic surfactants,
10% to 25% of glyceryl behenate,
0 to 10% of surfactants,
0 to 20% of binder(s),
0 to 10% of additives.

The anhydrous composition according to the invention may be in the various known galenical forms, which a person skilled in the art will adapt to the particular use of the composition.

The compositions according to the invention are preferably formulated for topical application.

The term “topical application” means external application to the skin or mucous membranes.

Via the topical route, the compositions may be in any galenical form normally used for topical administration. As non-limiting examples of topical compositions mention may be made of compositions as described in the American pharmacopoeias (USP32-NF27—Chap <1151>—Pharmaceutical Dosage Forms) or European pharmacopoeias (Edition 6.3 in the chapter: Préparations semi-solides pour application cutanée [Semi-solid preparations for cutaneous application]) or as defined in the decision trees of the American Food and Drug Administration (FDA) (CDER Data Standards Manual Definitions for topical dosage Forms). The compositions according to the invention may thus be in liquid, semi-solid, pasty or solid form, and more particularly in the form of ointments, oily solutions, dispersions of the lotion type, which may be two-phase lotions, serum, anhydrous or lipophilic gels, powders, impregnated pads, syndets, wipes, sprays, mousses, sticks, shampoos, compresses, washing bases, emulsions of liquid or semi-liquid consistency of the oil-in-glycol or glycol-in-oil type, a microemulsion, semi-liquid or solid suspensions or emulsions of the white or coloured cream type, gel or pomade, suspensions of microspheres or nanospheres or of lipid or polymeric vesicles, or microcapsules, microparticles or nanoparticles, or polymeric or gelled patches for controlled release.

In one preferred mode according to the invention, the composition is an anhydrous pharmaceutical or cosmetic composition of ointment type. The FDA thus defines an ointment as being a semi-solid composition comprising, as vehicle, less than 20% water and volatile compounds and more than 50% hydrocarbons, waxes or polyols. In certain cases, when the content of volatiles is high, such compositions may be referred to as creams (decision tree of the American Food and Drug Administration (FDA)). The American Pharmacopoeia defines an ointment as being a product whose base is a vehicle that may belong to the following four classes: hydrocarbon base or absorbent base or water-washable base or water-soluble base. The European Pharmacopoeia defines an ointment as being a one-phase composition in which liquids or solids may be dispersed.

The ointment according to the invention is preferentially a composition that is thick at room temperature, which comprises between 80% and 98% by weight, relative to the total weight of the composition, of hydrophobic compounds other than petroleum jelly. Such compounds are chosen especially from liquid oils alone or as a mixture, the said oils possibly being hydrocarbons, esters, plant oils and/or silicone oils, which are volatile or non-volatile, which may be gelled with lipophilic compounds that are solid at room temperature such as waxes, butters or fatty acid esters.

Optionally, a measurement of the flow threshold may be performed in order to characterize the finished product.

For the measurement of the flow threshold, a VT550 Haake rheometer with an SVDIN measuring spindle was used.

The rheograms are produced at 25° C. at an imposed speed of 0 to 100 s⁻¹. The viscosity values are given at shear values of 4 s⁻¹, 20 s⁻¹, 100 s⁻¹ (γ). The term “flow threshold” (τ₀ expressed in Pascals) means the force (minimum shear stress) required to overcome the cohesion forces of Van der Waals type and to bring about flow.

In one more particularly preferred mode, the composition comprises:
an active phase comprising, as pharmaceutical active agent, the phenolic derivative and at least one solvent for the phenolic derivative,
a non-active phase containing at least one fatty-phase thickener chosen from glyceryl behenate and derivatives and optionally an additional lipophilic thickener, and/or at least one oil and/or at least one lipophilic surfactant, and/or a binder, and/or any optional additive.

In another preferred mode according to the invention, the composition comprises:
an active phase comprising, as pharmaceutical active agent, the phenolic derivative and at least one solvent for the phenolic derivative,
a non-active phase containing at least one fatty-phase thickener chosen from glyceryl behenate and derivatives and optionally an additional lipophilic thickener, and/or at least one oil, and/or at least one lipophilic surfactant, and/or a binder, and/or any optional additive,
a polyorganosiloxane elastomer.

A subject of the invention is also the use of the composition thus obtained, as a medicament.

More particularly, the composition may be used for preparing a medicament intended for treating and preventing hyperpigmentary disorders such as melasma, chloasma, lentigo, senile lentigo, vitiligo, freckles, post-inflammatory hyperpigmentations caused by abrasion, a burn, a scar, a dermatosis or a contact allergy; naevi, genetically determined hyper-pigmentations, hyperpigmentations of metabolic or medicinal origin, melanomas or any other hyperpigmentary lesions.

The compositions according to the invention also find an application in the cosmetics field, in particular in protecting against the harmful effects of sunlight, for preventing and/or combating light-induced or chronological ageing of the skin and the integuments.

The invention also relates to a non-therapeutic cosmetic treatment process for beautifying the skin and/or for improving its surface appearance, characterized in that a composition comprising at least one depigmenting agent is applied to the skin and/or its integuments.

The anhydrous compositions according to the invention are obtained by a person skilled in the art using a known standard process for mixing phases.

The preparation process may especially include the following steps:
preparation of the active phase by incorporating the active agent into its fatty solvent, by means, if necessary, of heating;
preparation of the non-active phase(s);
incorporation of the active and non-active phases with stirring.

The formulation examples below illustrate the compositions according to the invention without, however, limiting the scope thereof. The amounts of the constituents are expressed as weight percentages relative to the total weight of the composition.

### EXAMPLE 1

### Study of Solubility/Stability of the Active Agents

a) Solubilities and Stabilities of Hydroquinone in Solvent Oils and Lipophilic Surfactants

Hydroquinone Solubilities

Hydroquinone solubilities

| Solvent | Max. solubility (w/w) | Method |
|---|---|---|
| Oily solvents | | |
| Caprylic capric | 1.7 | HPLC |
| triglycerides | | |
| (Miglyol ®812N) | | |
| Diisopropyl Adipate | 10.7 | HPLC |
| (Crodamol DA) | | |
| PPG-15 stearyl ether | 11.9 | HPLC |
| (Arlamol E) | | |
| Castor oil | 6.5 | HPLC |
| Lipophilic | | |
| surfactants | | |
| PEG-35 castor oil | 27.9 | HPLC |
| (Cremophor EL) | | |
| Lauroglycol | 5.5 | HPLC |
| PEG-8 caprylic capric | 28.9 | HPLC |
| triglycerides | | |
| (Labrasol) | | |
| Alcoholic or glycolic | | |
| solvents | | |
| Propylene glycol | 15.6 | HPLC |
| PEG 400 | 19.8 | HPLC |
| Glycerol | 15.6 | HPLC |
| Ethanol/water (20/80) | 17 | HPLC |

The above table makes it possible to identify which solvents are the most solubilizing for the active agent, for optimum selection of the ingredients of the composition. However, the choice of solvent will also be made on the basis of the results of the stability of hydroquinone in these solvents.

A compromise between solubility and stability must be obtained, if necessary by means of a mixture of solvents.

Stability of Hydroquinone in Solvent Oils and Lipophilic Surfactants

Assay technique by HPLC against reference substance.

The initial time (T0) is considered as 100%

| | 1 MONTH | | | | 3 MONTHS | | | |
|---|---|---|---|---|---|---|---|---|
| | RT | | 40° C. | | RT | | 40° C. | |
| Excipients | % | Colour | % | Colour | % | Colour | % | Colour |
| Caprylic | 94.2 | Colourless | 95 | Colourless | 93.1 | Colourless | 95 | Colourless |
| capric | | | | | | | | |
| triglycerides | | | | | | | | |
| (Miglyol ® | | | | | | | | |
| 218N) | | | | | | | | |
| Diisopropyl | 96.5 | Colourless | 95.9 | Colourless | 97.6 | Colourless | 97.4 | Colourless |
| Adipate | | | | | | | | |
| (Crodamol DA) | | | | | | | | |
| PPG-15 | 95.9 | Colourless | 95.5 | Colourless | 95.9 | Colourless | 96.0 | Colourless |
| stearyl ether | | | | | | | | |
| (Arlamol E) | | | | | | | | |
| PEG-35 castor | 91.4 | Orange | 87.1 | Brown | 87 | Brown+ | 85.1 | Brown++ |
| oil | | | | | | | | |
| (Cremophor | | | | | | | | |
| EL) | | | | | | | | |
| PEG-8 | 95.4 | Colourless | 95.2 | Colourless | 94.6 | Colourless | 95.1 | Colourless |
| caprylic | | | | | | | | |
| capric | | | | | | | | |
| triglycerides | | | | | | | | |
| (Labrasol) | | | | | | | | |
| Propylene | 97.0 | Colourless | 91.8 | Yellow | 94.2 | Yellow | 88.8 | Dark |
| glycol | | | | | | | | yellow |
| PEG 400 | 93.4 | Pink | 91.4 | Orange | 89.0 | Brown | 91.8 | Brown |
| Glycerol | 95.0 | Yellow | 93.5 | Dark | 93.0 | Pink | 93.3 | Orange |
| | | | | yellow | | | | |
| Ethanol/water | 94.4 | Orange | 91.7 | Dark | 93.9 | Orange | 89.4 | Brown |
| (20/80) | | | | pink | | | | |

The above table makes it possible to evaluate the stability of hydroquinone in the various solubilizing agents identified previously.

Thus, it may be deduced therefrom that the preferred solvents are Crodamol DA, Arlamol E and Labrasol, which give the hydroquinone good chemical and physical stability (macroscopic observation of the colour), coupled with a good solubilising effect.

The use of such solvents may thus make it possible to dispense with any use of antioxidants.

It may be noted that despite a high solubility of hydroquinone in Cremophor EL, it shows macroscopic instability demonstrated by browning that becomes pronounced over time and with temperature. Cremophor may be used in limited amount to aid the dissolution of the hydroquinone, but preferably along with a hydroquinone-stabilizing solvent, for instance medium-chain triglycerides such as Miglyol® 218N.

Moreover, it may be noted that in the solvents used in the prior art US 2006/0 120 979, such as glycols, a colouration is observed at RT and at 40° C., which is evidence of instability of the hydroquinone in these solvents in the absence of antioxidants.

b) Solubilities and Stabilities of Rucinol in Solvent Oils and Lipophilic Surfactants

Rucinol Solubilities

Rucinol solubilities

| Solvent | Max. solubility (% w/w) | Method |
|---|---|---|
| Oily solvents | | |
| Caprylic capric | 47.8 | HPLC |
| triglycerides | | |
| (Miglyol ®218N) | | |
| Diisopropyl Adipate | 64.1 | HPLC |
| (Crodamol DA) | | |
| PPG-15 stearyl | 48.2 | HPLC |
| ether (Arlamol E) | | |
| Castor oil | 46.4 | HPLC |
| Lipophilic | | |
| surfactants | | |
| PEG-35 castor oil | 49.7 | HPLC |
| (Cremophor EL) | | |
| Lauroglycol | 52.8 | HPLC |
| PEG-8 caprylic | 60.9 | HPLC |
| capric | | |
| triglycerides | | |
| (Labrasol) | | |
| Alcoholic or | | |
| glycolic solvents | | |
| Propylene glycol | >67.7 | HPLC |
| PEG 400 | 61.6 | HPLC |
| Glycerol | 63.8 | HPLC |
| Ethanol | >75.5 | HPLC |

The solubility study performed showed that rucinol shows very good solubility in all the solvents tested. However, the optimum choice of solvent will also be made on the basis of the results of the stability of rucinol in these solvents.

Stability of Rucinol in Solvent Oils and Lipophlic Surfactants

Assay technique by HPLC against reference substance.

The initial time (T0) is considered as 100%.

The table below makes it possible to evaluate the stability of rucinol in the solubilizing agents identified previously.

T + 1 month (% LC)

| | RT | 40° C. |
|---|---|---|
| Caprylic capric | 91.2 | 99.8 |
| triglycerides (Miglyol) | | |
| PPG-15 stearyl ether | 95 | 94.4 |
| (Arlamol E) | | |
| PEG-35 castor oil | 96 | 96.2 |
| (Cremophor EL) | | |
| PEG-8 caprylic capric | 97.8 | 100.8 |
| triglycerides (Labrasol) | | |
| Ethanol | 100.4 | 105 |

On the basis of these results, the following compositions according to the invention were prepared.

For all the formulations, the physical stability is checked by macroscopic and microscopic observation of the formulation at room temperature, at 4° C. and at 40° C. after 1 month, 2 months and optionally 3 months.

The macroscopic observation makes it possible to ensure the physical integrity of the products and the microscopic observation makes it possible to check that there is no recrystallization of the dissolved active agent.

The chemical stability is checked by assaying the active agents by external calibration in HPLC and the results are expressed as a % of duplication relative to the value obtained at T0 or relative to the theoretical titre.

### EXAMPLE 2

| Phases | INCI name | Formulation % |
|---|---|---|
| A | Glyceryl behenate | 16 |
| | Cetearyl isononanoate | 10 |
| B | Caprylic capric triglycerides | 34 |
| | Hydroquinone | 0.5 |
| C | PPG-15 stearyl ether | 15 |
| | Hydroquinone | 1.78 |
| | DL-α-tocopherol | 0.05 |
| | Ascorbyl palmitate | 0.1 |
| D | PEG-8 caprylic capric | 6 |
| | triglycerides | |
| | Hydroquinone | 1.72 |
| E | ST Elastomer 10 | 14.85 |

### PROCEDURE OF EXAMPLE 2

### Phase A

Introduce the glyceryl behenate and the cetearyl isononanoate into the formulation beaker. Bring the mixture to 85° C. with slow stirring and maintain the stirring and heating until fully homogeneous.

Stop the heating and maintain the stirring.

### Phase B

In a separate beaker, dissolve the minor part of hydroquinone in the caprylic/capric triglycerides with magnetic stirring while heating at about 75° C.

### Phase C

In a separate beaker, dissolve the DL-α-tocopherol and the ascorbyl palmitate and the second part of the hydroquinone in PPG-15 stearyl ether with magnetic stirring while heating at about 75° C.

### Phase D

In a separate beaker, dissolve the third part of the hydroquinone in PEG-8 caprylic/capric triglycerides with magnetic stirring while heating at about 75° C.

### Phase E

In a separate container, weigh out the ST Elastomer 10.

Mixing:

At about 75° C., add the fully homogenized phase B with stirring using a Rayneri blender.

At about 55° C., add phases C and D and homogenize fully with stirring using a Rayneri blender.

At 40° C. maximum, add phase E with continued stirring, and homogenize for about 5 minutes.

Leave to cool with stirring to about 35° C.

Specifications at T0 of Example 2:

Macroscopic appearance: glossy white ointment

Microscopic appearance: absence of hydroquinone crystals

Haake profile (4 s⁻¹/20 s⁻¹/100 s⁻¹): 72/68/103

Physical Stability:

| | | T + 1 month | T + 2 months | T + 3 months |
|---|---|---|---|---|
| Macroscopic | RT | In accordance | In accordance | In accordance |
| appearance | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |
| Microscopic | RT | In accordance | In accordance | In accordance |
| appearance | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |

| | | | |
|---|---|---|---|
| Haake rheology | 222/197/205 | 149/168/183 | 232/215/221 |
| (4 s⁻¹/20 s⁻¹/100 s⁻¹) | | | |

Chemical Stability:

[symbol] HYDROQUINONE

| | |
|---|---|
| Stability | Time |

| conditions | T0 | T + 1 M | T + 2 M | T + 3 M |
|---|---|---|---|---|
| RT | 99.4 | 98.1 | 95 | 101 |
| 4° C. | NA | 99.8 | 97.5 | 99.8 |
| 40° C. | NA | 103.1 | 96 | 100.3 |

### EXAMPLE 3

| Phases | INCI name | Formulation % |
|---|---|---|
| A | PPG-15 stearyl ether | 15 |
| | Hydroquinone | 2 |
| B | Caprylic capric | 57 |
| | triglycerides | |
| C | Cyclomethicone | 20 |

### PROCEDURE OF EXAMPLES 3 AND 4

### Phase A

In the formulation beaker, dissolve the hydro-quinone in the solvent (PPG-15 stearyl ether) with magnetic stirring while heating at 75° C.

Stop the heating and maintain the stirring.

### Phase B

In a separate beaker, weigh out the caprylic capric triglycerides.

### Phase C

In a separate beaker, weigh out the cyclomethicone or the Cetiol SN PH.

Mixing:

Once dissolved, introduce phase B into phase A with magnetic stirring.

Next, add the silicone or the Cetiol SN PH to the mixture obtained previously.

Leave to stir until homogeneous.

Specifications at T0 of Example 3:

Macroscopic appearance: Clear solution

Microscopic appearance: Absence of hydroquinone crystals

Physical Stability:

| | | T + 1 month | T + 2 months | T + 3 months |
|---|---|---|---|---|
| Macroscopic | RT | In accordance | In accordance | In accordance |
| appearance | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |
| Microscopic | RT | In accordance | In accordance | In accordance |
| appearance | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |

Chemical Stability:

[symbol] HYDROQUINONE

| | |
|---|---|
| Stability | Time |

| conditions | T0 | T + 1 M | T + 2 M | T + 3 M |
|---|---|---|---|---|
| RT | 102.5 | 103.8 | 104 | 107 |
| 4° C. | NA | 102.2 | 105 | 107.5 |
| 40° C. | NA | 101.9 | 103.5 | 107 |

### EXAMPLE 4

| Phases | INCI name | Formulation % |
|---|---|---|
| A | PPG-15 stearyl ether | 15 |
| | Hydroquinone | 2 |
| B | Caprylic capric | 70 |
| | triglycerides | |
| C | Cetiol SN PH | 10 |

Specifications at T0 of Example 4:

Macroscopic appearance: Clear solution

Microscopic appearance: Absence of hydroquinone crystals

Physical Stability:

| | | T + 1 month | T + 2 months | T + 3 months |
|---|---|---|---|---|
| Macroscopic | RT | In accordance | In accordance | In accordance |
| appearance | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |
| Microscopic | RT | In accordance | In accordance | In accordance |
| appearance | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |

Chemical Stability:

[symbol] HYDROQUINONE

| | |
|---|---|
| Stability | Time |

| conditions | T0 | T + 1 M | T + 2 M | T + 3 M |
|---|---|---|---|---|
| RT | 105.9 | 105.4 | 107 | 110.5 |
| 4° C. | NA | 105.7 | 108.5 | 111 |
| 40° C. | NA | 104.9 | 106 | 108.5 |

### EXAMPLE 5

| Phases | INCI name | Formulation % |
|---|---|---|
| A | Glyceryl behenate | 15 |
| | Hydrogenated castor oil | 2 |
| | Caprylic capric | 27 |
| | triglycerides | |
| B | PPG-15 stearyl ether | 35 |
| | Hydroquinone | 2 |
| C | Cyclomethicone | 19 |

### PROCEDURE OF EXAMPLES 5 AND 6

### Phase A

Introduce the glyceryl behenate, the hydrogenated castor oil and the caprylic capric triglycerides into the formulation beaker. Bring the mixture to 85° C. with slow stirring. Stop the heating when the mixture is homogeneous and maintain the stirring.

### Phase B

In a separate beaker, dissolve the hydroquinone in the PPG-15 stearyl ether with magnetic stirring while heating at about 75° C.

### Phase C

In a separate beaker, weigh out the cyclomethicone.

Mixing:

At 60° C. maximum, with magnetic stirring, pour B onto A. Homogenize well.

At 40° C. maximum, with magnetic stirring, add C. Mix until homogeneous.

Specifications at T0 of Example 5:

Macroscopic appearance: Glossy white ointment

Microscopic appearance: Absence of hydroquinone crystals

Haake profile (4 s⁻¹/20 s⁻¹/100 s⁻¹): 397/244/256

Physical Stability:

| | | T + 1 month | T + 2 months | T + 3 months |
|---|---|---|---|---|
| Macroscopic | RT | In accordance | In accordance | In accordance |
| appearance | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |
| Microscopic | RT | In accordance | In accordance | In accordance |
| appearance | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |

| | | | |
|---|---|---|---|
| Haake rheology | 419/270/295 | 366/239/266 | 355/235/282 |
| (4 s⁻¹/20 s⁻¹/100 s⁻¹) | | | |

Chemical Stability:

[symbol] HYDROQUINONE

| | |
|---|---|
| Stability | Time |

| conditions | T0 | T + 1 M | T + 2 M | T + 3 M |
|---|---|---|---|---|
| RT | 105.1 | 105.7 | 106 | 104 |
| 4° C. | NA | 105.5 | 109 | 105 |
| 40° C. | NA | 106.9 | 106 | 113 |

### EXAMPLE 6

| Phases | INCI name | Formulation % |
|---|---|---|
| A | Glyceryl behenate | 15 |
| | Hydrogenated castor oil | 2 |
| | Caprylic capric | 25 |
| | triglycerides | |
| B | PPG-15 stearyl ether | 35 |
| | Hydroquinone | 4 |
| C | Cyclomethicone | 19 |

Specifications at T0 of Example 6:

Macroscopic appearance: Glossy white ointment

Microscopic appearance: Absence of hydroquinone crystals

Haake profile (4 s⁻¹/20 s⁻¹/100¹): 337/226/250

Physical Stability:

| | | T + 1 month | T + 2 months | T + 3 months |
|---|---|---|---|---|
| Macroscopic | RT | In accordance | In accordance | In accordance |
| appearance | | with the | with the | with the |
| | | specifications | specifications | specifications |
| | 4° C. | In accordance | In accordance | In accordance |
| | | with the | with the | with the |
| | | specifications | specifications | specifications |
| Microscopic | RT | In accordance | In accordance | In accordance |
| appearance | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |

| | | | |
|---|---|---|---|
| Haake Theology | 182/196/276 | 292/187/235 | 354/269/278 |
| (4 s⁻¹/20 s⁻¹/100 s⁻¹) | | | |

Chemical Stability:

[symbol] HYDROQUINONE

| | |
|---|---|
| Stability | Time |

| conditions | T0 | T + 1 M | T + 2 M | T + 3 M |
|---|---|---|---|---|
| RT | 103.6 | 101.3 | 96.25 | 99 |
| 4° C. | NA | 100.8 | 95.75 | 101.75 |
| 40° C. | NA | 103.5 | 95.75 | 96.25 |

### EXAMPLE 7

| Phases | INCI name | Formulation % |
|---|---|---|
| A | PPG-15 stearyl ether | 21 |
| | Rucinol | 5 |
| B | Caprylic capric | 54 |
| | triglycerides | |
| C | Cyclomethicone | 20 |

PROCEDURE OF EXAMPLE 7

### Phase A

Dissolve the rucinol in the PPG-15 stearyl ether in the formulation beaker with magnetic stirring.

### Phase B

In a separate beaker, weigh out the caprylic/capric triglycerides.

### Phase C

In a separate beaker, weigh out the cyclomethicone.

Mixing:

With magnetic stirring, pour B onto A. Homogenize well.

Still with magnetic stirring, add C. Leave to stir until the mixture is homogeneous.

Specifications at T0 of Example 7:

Macroscopic appearance: Clear solution

Microscopic appearance: Absence of rucinol crystals

Physical Stability:

| | | T + 1 month | T + 2 months |
|---|---|---|---|
| Macroscopic | RT | In accordance | In accordance |
| appearance | | with the | with the |
| | | specifications | specifications |
| | 40° C. | In accordance | In accordance |
| | | with the | with the |
| | | specifications | specifications |
| | 4° C. | In accordance | In accordance |
| | | with the | with the |
| | | specifications | specifications |
| Microscopic | RT | In accordance | In accordance |
| appearance | | with the | with the |
| | | specifications | specifications |
| | 40° C. | In accordance | In accordance |
| | | with the | with the |
| | | specifications | specifications |
| | 4° C. | In accordance | In accordance |
| | | with the | with the |
| | | specifications | specifications |

Chemical Stability:

[symbol] RUCINOL

| Stability | Time | | |
|---|---|---|---|
| conditions | T0 | T + 1 M | T + 2 M |
| RT | 99.8 | 101 | 97.6 |
| 4° C. | NA | 100.4 | 100.0 |
| 40° C. | NA | 99.6 | 103.0 |

1.-21. (canceled)

22. An anhydrous pharmaceutical composition comprising a phenolic compound pharmaceutical active agent selected from among hydroquinone, rucinol or lucinol and salts thereof, 4-hydroxyanisole, hydroquinone monoethyl ether and hydroquinone monobenzyl ether, with the proviso that said phenolic compound is dissolved in a fatty phase therefor.

23. The anhydrous pharmaceutical composition as defined by claim 22, comprising an oily phase that is a solvent for the active agent.

24. The anhydrous pharmaceutical composition as defined by claim 23, wherein the oily phase comprises an oily solvent and/or a lipophilic surfactant for the active agent.

25. The anhydrous pharmaceutical composition as defined by claim 23, wherein the oily solvent is selected from among esters and derivatives thereof, ethers and derivatives thereof, or caprylic/capric triglycerides and derivatives thereof, or a mixture thereof.

26. The anhydrous pharmaceutical composition as defined by claim 23, comprising a mixture of oily solvents.

27. The anhydrous pharmaceutical composition as defined by claim 26, comprising a solvent mixture of a maximum of 15% by weight of ether solvent or derivative thereof relative to the total weight of the composition.

28. The anhydrous pharmaceutical composition as defined by claim 24, comprising a PEG-8 caprylic/capric triglyceride lipophilic surfactant.

29. The anhydrous pharmaceutical composition as defined by claim 22, comprising an amount of antioxidant less than 0.3% by weight relative to the total weight of the composition.

30. The anhydrous pharmaceutical composition as defined by claim 22, devoid of any antioxidant.

31. The anhydrous pharmaceutical composition as defined by claim 22, further comprising at least one lipophilic thickener or gelling agent.

32. The anhydrous pharmaceutical composition as defined by claim 22, further comprising at least one additional fatty substance.

33. The anhydrous pharmaceutical composition as defined by claim 22, wherein the phenolic compound is present therein in an amount ranging from 0.01% to 10% by weight relative to the total weight of the composition.

34. The anhydrous pharmaceutical composition as defined by claim 22, wherein the phenolic compound comprises hydroquinone or rucinol or salt thereof.

35. The anhydrous pharmaceutical composition as defined by claim 22, devoid of any alcoholic or glycolic solvent.

36. The anhydrous pharmaceutical composition as defined by claim 22, devoid of any preservative.

37. The anhydrous pharmaceutical composition as defined by claim 31, comprising an additional lipophilic thickener or gelling agent selected from among waxes, fatty alcohols, hydrogenated oils and fatty acid esters.

38. The anhydrous pharmaceutical composition as defined by claim 37, comprising a glyceryl behenate and/or derivative thereof lipophilic thickener.

39. The anhydrous pharmaceutical composition as defined by claim 22, comprising an organopolysiloxane elastomer.

40. A medicament comprising an anhydrous pharmaceutical composition as defined by claim 22.

41. A medicament useful for treating and/or preventing hyperpigmentary disorders, melasma, chloasma, lentigo, senile lentigo, vitiligo, freckles, post-inflammatory hyperpigmentations due to abrasion, a burn, a scar, a dermatosis or a contact allergy; naevi, genetically determined hyperpigmentations, hyperpigmentations of metabolic or medicinal origin, melanomas or any other hyperpigmentary lesions, comprising an anhydrous pharmaceutical composition as defined by claim 22.

42. A medicament useful for protecting against the harmful effects of sunlight, for preventing and/or combating light-induced or chronological aging of the skin and integuments thereof, comprising an anhydrous pharmaceutical composition as defined by claim 22.
